(19) United States
(12) Patent Application Publication
(10) Pub. No.: US 2020/0222454 A1
(43) Pub. Date: Jul. 16, 2020

(54) NEW THERAPY FOR OSTEOARTHRITIC PAIN

(71) Applicant: MERCK PATENT GMBH, DARMSTADT (DE)

(72) Inventors: CHRISTOPH H. LADEL, DARMSTADT (DE); HANS GUEHRING, GEISENHEIM (DE)

(21) Appl. No.: 16/650,876

(22) PCT Filed: Sep. 26, 2018
(86) PCT No.: PCT/EP2018/076049

§ 371 Date: Mar. 26, 2020

(30) Foreign Application Priority Data
Sep. 27, 2017 (EP) 17193587.7

(51) Int. Cl. A61K 33/42; A61K 9/00; A61K 33/14; A61K 31/19; A61P 19/02

(52) U.S. Cl. CPC A61K 33/42; A61K 9/0019; A61P 19/02; A61K 31/19; A61K 33/14

(57) ABSTRACT

The present invention relates to the treatment of osteoarthritic pain and to the slowing down of the structural osteoarthritis progression in a patient having osteoarthritis. More particularly, it relates to a physiological compound for use according to particular treatment regimens. Specifically, it concerns a treatment scheme comprising weekly administration for three or four times of a physiological compound per treatment cycle.

### FIELD OF INVENTION

The present invention relates to the treatment of osteoarthritic pain and to the slowing down of the structural osteoarthritis progression in a patient having osteoarthritis. More particularly, it relates to a physiological compound for use according to particular treatment regimens. Specifically, it concerns a treatment scheme comprising weekly administration for three or four times of a physiological composition per treatment cycle.

### BACKGROUND OF THE INVENTION

Cartilage is composed of chondrocytes (cells derived from mesenchymal cells) which are dispersed in the matrix (a firm, gel-like ground substance). The cartilaginous matrix is produced by these cells and comprises mainly Type II collagen fibers (except fibrocartilage which also contains type I collagen fibers), proteoglycans, and elastin fibers. Cartilage is found among other places in the joints, the rib cage, the ear, the nose, in the throat, in the trachea and in the intervertebral disks. There are three main types of cartilage: hyaline, elastic and fibrocartilage, providing notably different functional properties according to their histological morphology. Articular cartilage, for instance, is a hyaline cartilage, having viscoelastic properties, covering the articular surfaces of bones. The main purpose of articular cartilage is to provide smooth surfaces in order to ensure nearly frictionless movement of articulating bones.

Cartilage disorders broadly refer to diseases characterized by degeneration/disintegration of cartilage and abnormalities in the connective tissues which are manifested by inflammation, pain, stiffness and limitation of motion of the affected body parts. These disorders can be due to a pathology or can be the result of trauma or injury. Mature cartilage has very limited ability to self-repair, notably because mature chondrocytes have little potential for proliferation due to limited supply with nutrients linked to the absence of blood vessels in cartilage. Replacement of damaged cartilage, in particular articular cartilage, caused either by injury or disease, is a major challenge for physicians, and available surgical treatment procedures are considered unpredictable and effective for only a limited time in younger patients without osteoarthritic changes. Therefore, the majority of patients either do not seek treatment or are counselled to postpone treatment for as long as possible. When treatment is required, the standard procedure is age dependent and varies between total or partly joint replacement, transplantation of pieces of cartilage or chondrocytes or marrow stimulating technique (such as microfracture). Microfracture is a cheap and common procedure that involves penetration of the subchondral bone to stimulate cartilage deposition by bone marrow derived stem cells. However, it has been shown that this technique does not repair sufficiently the chondral defect and the new cartilage formed is mainly fibrocartilage, resulting in a short-lived repair tissue. Indeed, fibrocartilage does not have the same biomechanical properties as hyaline articular cartilage and lacks often proper lateral integration into the surrounding cartilage. For this reason, the newly synthesized fibrocartilage may breakdown more easily (expected time frame: 5-10 years).

For patients with osteoarthritis (OA) all these cartilage repair techniques fail. The remaining non-surgical treatment consists notably of physical therapy, lifestyle modification (e.g. body weight reduction), supportive devices, oral drugs (e.g. non-steroidal anti-inflammatory drugs), injection of drugs (e.g. hyaluronic acid and corticoids), and food supplementation. All these treatments are unable to stop OA disease progression. If the pain therapy also fails, surgery, such as joint replacement or high tibial osteotomy for the knee joint, is the only remaining option for the patients. Tibial or femoral osteotomies (cutting the bone to rebalance joint wear) may reduce symptoms, help to maintain an active lifestyle, and delay the need for total joint replacement. Total joint replacement can provide relief for the symptom of advanced osteoarthritis, but generally requires a significant change in a patient's lifestyle and/or activity level.

Current available drug treatments are mainly directed to pain relief. At this time, there is no commercially available treatment that restores the cartilage damages (see Karsdal, 2016). The effect on pain however is limited, and usual provide relief only for limited time or are limited due to adverse events and side effects (Strand 2011). Also, effect sizes on pain in OA clinical studies are largely influenced by placebo effects. This placebo effect is already known in OA (Altman 2016; Bannuru, 2015).

Therefore, there is a need of an alternative treatment for decreasing pain/improving function for a longer period (e.g. slowing down the structural progression of the disease). Indeed, pain is not only very often associated with cartilage disorders but represents the leading symptom for clinical detection of these disorders.

### SUMMARY OF THE INVENTION

It is an object of the present invention to provide a method for treating osteoarthritic pain in a patient having osteoarthritis comprising the administration of a physiological compound wherein the physiological compound is administered at least three times per treatment cycle, said administrations being separated by about 1 week, preferably by 1 week (i.e. weekly administrations). In a preferred embodiment said administrations are separated by regular interval of 1 week each.

The present invention further provides a physiological compound for use in the treatment of osteoarthritic pain in a patient having osteoarthritis wherein the physiological compound is to be administered at least three times per treatment cycle, said administrations being separated by about 1 week, preferably by 1 week (weekly administrations). In a preferred embodiment said administrations are separated by regular interval of 1 week each.

It is a further object of the present invention to provide a method for slowing down the structural osteoarthritis progression in a patient having osteoarthritis comprising the administration of a physiological compound wherein the physiological compound is administered at least three times per treatment cycle, said administrations being separated by about 1 week, preferably by 1 week (i.e. weekly administrations). In a preferred embodiment said administrations are separated by regular interval of 1 week each.

The present invention further provides a physiological compound for use in slowing down the structural osteoarthritis progression in a patient having osteoarthritis wherein the physiological compound is to be administered at least three times per treatment cycle, said administrations being separated by about 1 week, preferably by 1 week (weekly administrations). In a preferred embodiment said administrations are separated by regular interval of 1 week each.

Preferably, the physiological compound to be administered is selected from the group consisting of saline, phosphate-buffered saline (PBS), sodium lactate solution or combination of saline with PBS.

### Definitions

The term “physiological compound”, as used herein, is intended to be a compound selected from the group consisting of saline (alternatively called sodium chloride, NaCl, physiologic saline or isotonic saline), phosphate-buffered saline (PBS) or combination of both saline with PBS. PBS is a water-based salt solution containing disodium hydrogen phosphate, sodium chloride and, in some formulations, potassium chloride and/or potassium dihydrogen phosphate. It is used as a buffer to maintain a constant pH. The osmolarity and ion concentrations of the solutions match those of the human body (isotonic). Non-limiting examples of PBS solutions that can be used in the context of the present invention are the following: 1) 140 mMol NaCl, 10 mMol Na2HPO4, 2 mMol KH2PO4, 3 mMol KCl, pH 7.4 or 2) 7 mM Na2HPO4, 1 mM KH2PO4 and 2.7 mM KCL, pH 7.4. Sodium lactate solution, also known as Ringer's lactate solution (RL) or Hartmann's solution, is a mixture of sodium chloride, sodium lactate, potassium chloride, and calcium chloride in water. A non-limiting example of such RL solution that can be used in the context of the present invention is the following: 130 mMol Sodium, 5 mMol Potassium, 1.5 mMol Calcium, 110 mMol Chloride, 28 mMol Lactate, pH 5.0-7.0.
The term “about” in “about one week” or “about every week” encompasses administrations separated by one week (7 days), as well as administration separated by one week+/−few days (e.g. +/−1 or 2 day(s)). Indeed, it should be understood that, notably for a practical point of view, the administration of the physiological compound cannot always be performed at exact intervals, e.g. exactly one week (7 days) day per day after the previous administration. Therefore, in the context of the invention, one week means 7 days, but may also be 5, 6, 8 and 9 days after the previous administration, for the convenience of the patient. In the context of the present invention, the term “one week” is similar to the terms “every week” or “weekly” and they can be used interchangeably. “Week” can be used should one refers to “days” (e.g. 1^{st} injection a Monday, following injection a Monday one week after) or should one refers to a “date” (e.g. 1^{st} injection the 1^{st} of September. Following injection the 8^{th} of September).
The term “treatment cycle” or “cycle” corresponds to the period wherein an physiological compound is given every week (consecutive administrations). As an example, a treatment cycle can consist of three injections at one week interval each. Such a “treatment cycle” can be repeated. For instance, a second “treatment cycle” can be performed 2, 3, 4, 5 or 6 months after the last injection of the previous cycle. Alternatively, a second cycle can also be performed one year or two years after the first injection in the first cycle. As an example, a first treatment cycle consisting of three injections with one week interval each can be followed, six months after the last injection of said cycle, by a second treatment cycle of three injections with one week interval each.
The term “synovial aspiration” refers to a procedure to remove fluid from the space in a joint using a needle and syringe. This is usually done under a local anesthetic either to relieve swelling or to obtain fluid for analysis to diagnose a joint disorder and/or problem.
The term “Osteoarthritis”, or “OA”, is used to intend the most common form of arthritis. The term “osteoarthritis” encompasses both primary osteoarthritis and secondary osteoarthritis (see for instance The Merck Manual, 17th edition, page 449). Osteoarthritis may be caused by the breakdown of cartilage. Bits of cartilage may break off and cause pain and swelling in the joint between bones. Over time, the cartilage may wear away entirely, and the bones will rub together. Osteoarthritis can affect any joint but usually concerns hands, shoulders and weight-bearing joints such as hips, knees, feet, and spine. In a preferred example, the osteoarthritis may be knee osteoarthritis or hip osteoarthritis. This wording encompasses notably the forms of osteoarthritis which are classified as stage 1 to stage 4 or grade 1 to grade 6 according to the OARSI classification system. The skilled person is fully aware of osteoarthritis classifications that are used in the art, in particular said OARSI assessment system (also named OOCHAS; see for instance Custers et al., 2007).
In the context of the present invention, the “efficacy” of a pain treatment can be measured based on WOMAC changes.
“WOMAC total scores” or “WOMAC scores” (“WOMAC” for “Western Ontario and McMaster Universities Osteoarthritis Index”) measure pain (WOMAC pain score), function (WOMAC function score) and stiffness (WOMAC stiffness score). When applied to assessment of pain and dysfunction associated with osteoarthritis, it consists of a questionary containing 24 items divided into 3 subscales (5 items for Pain, 2 items for Stiffness and 17 items for Physical Function)(see Bellamy et al., 1988; Wolfe, 1999). It is a well-known instrument, widely used notably in assessment of the OA severity.
The term “baseline” means before treatment (i.e. at study entry). In the context of the present invention it refers notably to WOMAC total score of one given subject at study entry (i.e. before treatment with physiological compound).
The term “slowing down the structural osteoarthritis progression” in a patient having osteoarthritis means loss of cartilage as measured by imaging technologies like MRI and/or x-ray determined using e.g., but exclusively, cartilage thickness, cartilage volume and/or joint space width measurements as read-outs. Progression in general is described to be more 0.01 mm per year cartilage thickness loss (With rates of about −1 to −3% and standardized response means of −0.3 to −0.5 per year; Hunter, 2009; Hunter, 2011) determined by MRI or 0.13 mm joint space narrowing/year determined by x-ray (Emrani, 2008). Alternatively, the term “slowing down of further cartilage loss)” in a patient having osteoarthritis can be used.
In the context of the present invention, the “efficacy” of a treatment, in term of slowing down the structural OA progression can be measured based on MRI changes.
The term “subject” or “patient” refers to both human and non-human animals. The term non-human comprises mammals such as rodents (including mice), rabbits, cats, dogs, horses, cows, sheep, or primates.
The “response”, or “sensitivity” to a physiological compound treatment is to be understood as at least at 6-month or even better one year after the first injection and measured as decrease of WOMAC total scores, or changes in WOMAC total scores.

### DETAILED DESCRIPTION OF THE INVENTION

The present invention provides administration schemes for the treatment of osteoarthritic pain with a physiological compound. Preferably, said physiological compound is selected from the group consisting of saline, phosphate-buffered saline, sodium lactate solution/Ringer Lactate solution or combination of both saline and PBS. Preferably, when saline is used, it is at or at about 0.9% w/v. In the context of the present invention it has been surprisingly shown by the inventors that a physiological compound had optimal symptom-ameliorating effects on osteoarthritic pain and on slowing down the structural osteoarthritis progression when administered according to the methods and uses disclosed herein. It has been surprisingly found that the dosing regimen of the present invention (i.e. once weekly for three week per treatment cycle) cause durable reduced pain and slowing down the structural osteoarthritis progression (in other words a slowing down of further cartilage loss), both for at least 12 months after first injection. This finding was not expected because of the rather short half-life of the composition as described, and the absence of active ingredients in the composition.

In one embodiment, the present invention provides a method for treating osteoarthritic pain, in a patient having osteoarthritis, comprising the administration of a physiological compound wherein the physiological compound is administered at least three times per treatment cycle, said administrations being separated by about one week, preferably by one week (i.e. every weeks or weekly).

In another aspect of the present invention, here is provided a physiological compound for use in the treatment of osteoarthritic pain, in a patient having osteoarthritis, wherein the physiological compound is administered at least three times per treatment cycle, said administrations being separated by about one week, preferably by one week (i.e. every weeks or weekly).

It is a further aspect of the present invention to provide a method for slowing down the structural osteoarthritis progression (or in other words a method for slowing down of further cartilage loss) in a patient having osteoarthritis comprising the administration of a physiological compound wherein the physiological compound is administered at least three times per treatment cycle, said administrations being separated by about 1 week, preferably by 1 week (i.e. weekly administrations). In a preferred embodiment said administrations are separated by regular interval of 1 week each.

In an even further aspect, the present invention further provides a physiological compound for use in for slowing down the structural osteoarthritis progression (or in other words a method for slowing down of further cartilage loss) in a patient having a osteoarthritis wherein the physiological compound is to be administered at least three times per treatment cycle, said administrations being separated by about 1 week, preferably by 1 week (weekly administrations). In a preferred embodiment said administrations are separated by regular interval of 1 week each

In the context on the present invention as a whole, any treatment (or any method for treating) with a physiological compound, or any use if a physiological compound, can further comprise synovial aspiration. Preferably, such aspiration is performed just before physiological compound injection or concomitantly with the administration of the physiological compound.

According to the present invention as a whole, the administration of the physiological compound, either alone or in combination with synovial aspiration, is to be performed at regular intervals, however slight variations of +/−few days are authorized (preferably not more than two days). For example, where administrations are separated by about one week, if the first administration of a cycle is given on a Wednesday, the second administration may be made the Wednesday one week after the first administration (regular interval) or a few days after or before (for instance the Monday before or Thursday after). Similarly, if the first administration is given for instance the 1^{st} of September, the second administration may be made the 8^{th} of September, i.e. one week after the first administration (regular interval) or a few days after or before (for instance the 7^{th} of September or the 9^{th} of September). Such a flexibility allows the dosing regimen to be notably less restricting and more convenient for the patient. Preferably, administrations are performed on a regular interval basis, e.g. every week. In one preferred embodiment they are separated by one week (i.e. weekly injection).

In the context of the present invention as a whole, the physiological compound is administered at least three times per treatment cycle. It can also be administered, for instance, at least four times per treatment cycle. Preferably, it is administered for three times or four times per treatment cycle.

In a preferred embodiment, the physiological compound can be administered three times per treatment cycle, in regular intervals of one week or of about one week (i.e. three times per treatment cycle, in regular intervals of one week or once every week).

In a preferred embodiment, the physiological compound, either alone or in combination with synovial aspiration, is to be administered for at least three consecutive times or at least four consecutive times per treatment cycle. In a further preferred embodiment the physiological compound, either alone or in combination with synovial aspiration, is administered for three consecutive times or four consecutive times per treatment cycle. In an even preferred embodiment, it is administered for three consecutive times.

In the context of the present invention as a whole, such treatment may comprise several treatment cycles per year, such as 1, 2 or 3 treatment cycles per year. In one preferred embodiment, such treatment comprises 2 cycles per year. As an alternative, the treatment comprises 1 cycle per year, repeated 1 year or 2 years after the beginning of the first treatment cycle. As an example, should a treatment comprising 1 cycle, said treatment may consist of 3 injections at 1 week interval each. As a further example, should a treatment comprising at least 2 cycles, a first treatment cycle consisting of 3 injections at 1 week interval each can be followed, several months after the last injection of said cycle, by a second treatment cycle of 3 injections at 2 weeks interval each.

The physiological compound of the invention is preferably selected from the group consisting of saline, phosphate-buffered saline (PBS), sodium lactate solution or combination of both saline with PBS. Preferably, when saline is used, either alone or in combination with PBS, as the physiological compound, it is at or at about 0.9% w/v. When PBS is used as the physiological compound, alone or in combination with saline, said PBS solution can be for instance made of 1) 140 mMol NaCl, 10 mMol Na2HPO4, 2 mMol KH2PO4, 3 mMol KCl, pH 7.4 or 2) 7 mM Na2HPO4, 1 mM KH2PO4 and 2.7 mM KCL, pH 7.4. When a sodium lactate solution is used as the physiological compound, said solution can be for instance made of 130 mMol Sodium, 5 mMol Potassium, 1.5 mMol Calcium, 110 mMol Chloride, 28 mMol Lactate, in water, pH 5.0-7.0. Said physiological compound decreases osteoarthritic pain and slows down the structural disease progression.

In a further preferred embodiment, the treatment comprises administration of the physiological compound at a volume of or of about 1 to 10 mL (such as 1 to 10 mL of a physiological compound comprising 0.9% w/v saline in PBS per single intra-articular administration. In a preferred embodiment the treatment comprises administration at a volume of or of about 1 to 5 mL (such as 1, 2, 3, 4 or 5 mL) per single intra-articular administration of the physiological compound. Preferred volumes include 1 to 3 mL, such as 1.5, 1.6, 1.7, 1.8, 1.9, 2.0, 2.1, 2.2, 2.3, 2.4 or 2.5 mL per single intra-articular administration of the physiological compound. It should be understood that the volume of the physiological compound to be administered will be different should the patient to be treated is a human or a non-human mammal. For instance, for dogs, the dose will be preferably 5-fold less important than for human. As an example, should the human volume being range from 1 to 10 mL per single intra-articular administration, the volume for a dog could be ranged from 0.2 to 2 mL per single intra-articular administration. The preferred posology cycle for a human patient is 2 ml of a physiological composition (e.g. comprising 0.9% w/v saline in PBS) per intra-articular injection, once weekly for 3 consecutive administrations (one treatment cycle). The physiological compound does not need to be further formulated as a pharmaceutical composition, i.e. together with a pharmaceutically acceptable carrier, excipients or the like. For parenteral or intraarticular administration, the physiological compound may be formulated in a unit dosage form for injection. It needs to be sterile and compatible with the physiological conditions at the application site (e.g., knee joint, synovial fluid).

In a preferred embodiment, the treatment comprises intra-articular administration of the physiological compound, either alone or together with synovial aspiration. The physiological compound can be applied by direct injection into the synovial fluid of the joint or directly into the defect. Preferably, the mode of administration of the physiological compound is selected from the group consisting of: intra-synovial administration and intra-articular administration. The intraarticular administration is done in a joint selected from joint of the hip, knee, elbow, wrist, ankle, spine, feet, finger, toe, hand, shoulder, ribs, shoulder blades, thighs, shins, heels and along the bony points of the spine. In yet another preferred embodiment the intraarticular administration is done in the joint of the hip or the knee.

### DESCRIPTION OF THE FIGURES

FIG. 1: Design of the study with injection schemes and schedules of assessments.

FIG. 2: A) Effect of composition injection cycles (arrows) on total WOMAC score up to week 156. B) Lower panel: effect of composition injection cycles (arrows) on total cartilage thickness in the total femorotibial joint of the target knee.

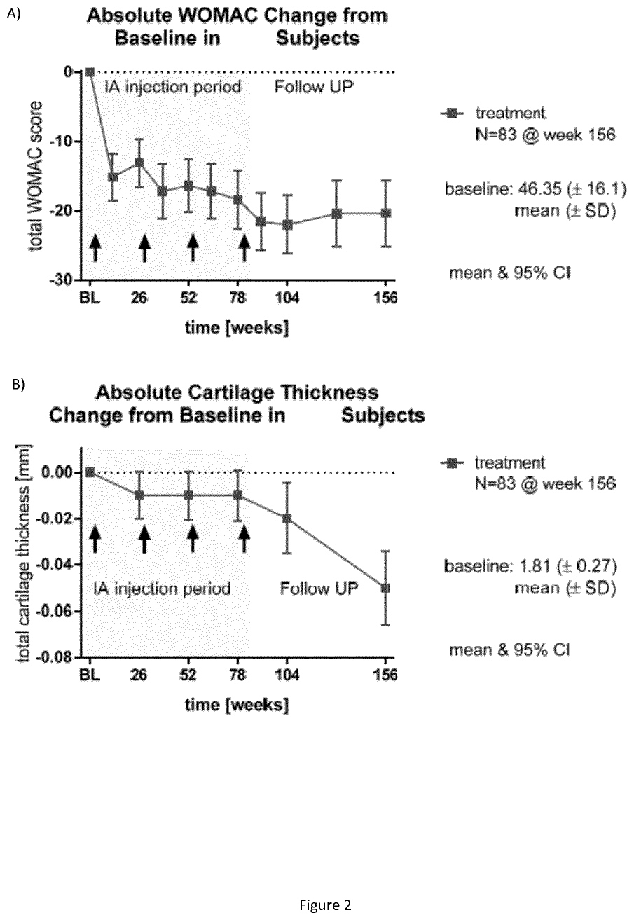

### EXAMPLES

Materials

The physiological compound used was composed of 0.9% w/v saline in PBS composed of 7 mM Na2HPO4, 1 mM KH2PO4 and 2.7 mM KCL. It was used at a volume of 2 ml. 1 glass ampule of sterile composition solution for injection (2 mL per ampule) was used for all treatment groups. The whole volume was administered to each patient.

Methods

Dosing Regimen

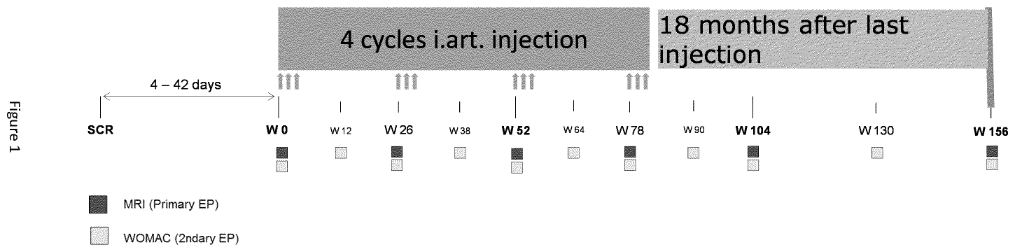

The patients received 4 cycles of treatment (each consisting of three once-weekly intra articular injections of the physiological compound) over three consecutive weeks at intervals of 6 months (see FIG. 1).

Inclusion/Exclusion Criteria

The study included adult subjects of either sex with primary femorotibial OA according to American College of Rheumatology (ACR) clinical and radiographic criteria who had Kellgren-Lawrence grades (KLG) of 2 or 3. Subjects must have had pain in the target knee on most days and/or require symptomatic treatment of knee pain with paracetamol (acetaminophen), systemic non-steroidal anti-inflammatory drugs (NSAIDs) including COX inhibitors (COXibs), or tramadol on most days of the previous month, and must have had both: 1) A history of pain due to OA in the target knee for at least 6 months, and 2) Pain score for the target knee of 4 to 9 points in response to Question 1 of the Western Ontario and McMaster Universities Osteoarthritis Index (WOMAC) pain index (“how much pain have you had [in the target knee, over the past 48 hours] when walking on a flat surface?”) at screening and Baseline, after washout of at least 5 half-lives of analgesic medication(s): acetaminophen, topical or oral systemic NSAIDS, COXibs, opioids, and/or tramadol. Women of childbearing potential must have used a form of contraception with a failure rate of less than 1% per year throughout the study.

Main exclusion criteria included malalignment of >5 degrees in the femorotibial axis of the target knee, clinical signs of inflammation (i.e. redness) in the target knee, i.art. administration of corticosteroids or hyaluronic acid into either knee within 6 months before screening, any plan for knee surgery (affecting either the target or the contralateral knee) within the next 2 years, concomitant conditions or treatments deemed to be incompatible with study participation, contraindications to MRI scanning (including inability to fit in the scanner or knee coil), pregnancy or breastfeeding, participation in another clinical study within the past 30 days, and legal incapacity or limited legal capacity.

Screening Period

The screening period comprised a period of 4 to 42 days during which a subject's eligibility for the study was determined, beginning at signature of the ICF (Visit 1a). Subjects who had taken analgesia within 5 half-lives of the visit were scheduled to return for complete screening procedures (Visit 1b) after a washout of all analgesic medications. Subjects not taking analgesic medication at the time of ICF signature could have completed Visit 1a and 1b procedures at 1 visit. Screening included completion of Question 1 of the Western Ontario and McMaster Universities Osteoarthritis Index (WOMAC) pain index and pain on an 11-point pain numerical rating scale (NRS) in both the target and contralateral knee. Subjects who had a WOMAC Question 1 score in the target knee of 4 to 9 after a predefined analgesic washout period equivalent to at least 5 half-lives of the analgesic medication(s) continued with other screening procedures.

Included Patients

Overall n=107 patients were included. At week 156 still n=83 patients were examined.

Endpoint Assessments:

One of the endpoint was changes from baseline in the WOMAC total score and in the WOMAC pain, function, and stiffness index scores over 3 years. The questionnaire addresses the degree of pain experienced with different activities or positions (5 questions), the degree and timing of joint stiffness (2 questions), and the degree of difficulty experienced in performing daily activities (17 questions).

The maximum possible total score, indicating the worst possible OA symptoms, is 240 points. For convenience and ease of interpretation, all scores were normalized on scales of 0 to 100 points. The minimum clinically important difference in WOMAC scores is considered to be about 10% of the maximum possible score.

WOMAC and other questionnaires were filled-in on week 0, 12, 26, 38, 52, 64, 78, 90, 104. Structural changes in cartilage thickness in the total femorotibial joint of the target knee were assessed in terms of imaging by magnetic resonance imaging (MRI).

Results

Even after the first injection a decrease in total WOMAC score was demonstrable, thereby demonstrating clinical benefit for the patients, i.e. feeling less pain. This decrease in WOMAC score compared to baseline was sustained after repeated injections and even sustained up to 18 months after the last injection.

Also the total cartilage thickness was assessed and during the injection period (up to week 78) the loss of cartilage was decreased. In the follow-up after the injection period a more progressive loss of cartilage was demonstrable using MRI readings at different time points.

### CONCLUSIONS

The results obtained in the study demonstrated symptomatic benefit as well as slow structural progression during intra-articular injection of the composition. The symptomatic benefit as demonstrated by decrease of the total WOMAC score was sustained at least up week 156, i.e. more than 18 months after last injection.

### REFERENCES

[1] Altman et al, 2016, Seminars in Arthritis and Rheumatism, 46:151-159
[2] Bannuru et al., 2015, Ann Intern Med 163: 365-372
[3] Karsdal et al. 2016, Osteoarthritis Cartilage. 2016 December; 24(12):2013-2021
[4] Hunter et al., 2011, Osteoarthritis Cartilage 19: 589-605
[5] Hunter et al., 2009, Current Opinion in Rheumatology 21:110-117
[6] Emrani et al. 2008, Osteoarthritis Cartilage 16: 873-882
[7] Strand et al., 2011, Osteoarthritis Cartilage 19: 493-499

1-12. (canceled)

13. A method of treating osteoarthritic pain in a patient having osteoarthritis comprising the administration of a physiological compound, wherein the physiological compound is administered at least three times per treatment cycle, said administrations being separated by about 1 week.

14. The method according to claim 13, wherein said administrations are separated by 1 week.

15. The method according to claim 13, wherein the physiological compound is administered at least 3 times or at least 4 times per treatment cycle.

16. The method according to claim 13, wherein treatment cycles are repeated after 2, 3, 4, 5 or 6 months.

17. The method according to claim 13, said method comprising 1, 2, or 3 treatment cycles per year.

18. The method according to claim 13, wherein the physiological compound is administered intra-articularly.

19. The method according to claim 13, wherein the physiological compound is administered after a step of synovial aspiration.

20. The method according to claim 13, wherein the physiological compound is administered at a volume of about 1-5 ml, about 1 to 3 ml, or about 2 ml per single intra-articular administration.

21. The method according to claim 13, wherein the physiological compound is to be administered at a volume of about 1, 2, 3, 4 or 5 mL per single intra-articular administration of the physiological compound.

22. The method according to claim 13, wherein the physiological compound is selected from the group of saline, phosphate-buffered saline (PBS), sodium lactate solution or combination of both saline and PBS.

23. The method according to claim 22, wherein saline is at or at about 0.9% w/v.

24. A method of slowing structural osteoarthritis progression in a patient having osteoarthritis comprising the administration of a physiological compound, wherein the physiological compound is administered at least three times per treatment cycle, said administrations being separated by about 1 week.

25. The method according to claim 24, wherein said administrations are separated by 1 week.

26. The method according to claim 24, wherein the physiological compound is administered at a volume of about 1-5 ml, about 1 to 3 ml, or about 2 ml per single intra-articular administration.

27. The method according to claim 24, wherein the physiological compound is to be administered at a volume of about 1, 2, 3, 4 or 5 mL per single intra-articular administration of the physiological compound.

28. The method according to claim 24, wherein the physiological compound is selected from the group of saline, phosphate-buffered saline (PBS), sodium lactate solution or combination of both saline and PBS.
